(19) United States
(12) Patent Application Publication
(10) Pub. No.: US 2021/0346923 A1
(43) Pub. Date: Nov. 11, 2021

(54) METHODS AND PRODUCTS FOR BIODEGRADATION OF WASTE

(71) Applicant: Westcom Solutions Pte. Ltd., Singapore (SG)

(72) Inventors: Qingxin Li, Singapore (SG); Jinchuan Wu, Singapore (SG); David Tan, Singapore (SG); Yeru Fang, Singapore (SG)

(21) Appl. No.: 16/869,037

(22) Filed: May 7, 2020

(51) Int. Cl. B09B 3/00; C12P 7/56; C12P 7/54; C12N 11/02

(52) U.S. Cl. CPC B09B 3/00; C12P 7/56; C12N 1/20; C12N 11/02; C12P 7/54

(57) ABSTRACT

The present invention provides a method of producing a product for waste degradation, a microbial consortium or a mixed microbial composition, a product for waste degradation, and a waste degradation method. The method of producing the product for waste degradation comprises providing at least one isolated microbial strain from excreta, providing at least one lactic acid and/or acetic acid producing microbial strain, providing at least one yeast strain, providing at least one Bacillus sp., and combining the microbial strains to produce a microbial consortium for waste degradation.

### SEQUENCE LISTING

The instant application contains a Sequence Listing which has been submitted in ASCII format via EFS-Web and is hereby incorporated by reference in its entirety. Said ASCII copy, created on May 7, 2020, is named WTC 0001PA_SP102588US SEQ LISTING_ST25.txt and is 1 KB in size.

### TECHNICAL FIELD

The present invention relates to waste biodegradation in general and more particularly to a method of producing a product for waste degradation, a microbial consortium or a mixed microbial composition for waste degradation, a product incorporating the same and a waste degradation method.

### BACKGROUND

The amount of waste generated by the human population continues to increase at an alarming rate. Almost any human activity would generate waste. Such waste can be in the solid, liquid or gaseous form. Waste is generated by any living organism.

Human waste includes municipal waste, sewage waste and industrial waste for example. Proper waste management is of increasing importance and there are many factors, aspects and goals to consider; such as reduction/removal of toxic and hazardous materials. Biodegradation of organic waste is an important arm of the waste management spectrum.

In areas where toilet sanitation with a sewerage system and waste treatment may not be available, it is desirable to develop efficient composting methods and products for excreta waste.

### SUMMARY

According to a first aspect, the present invention provides a method of producing a product for waste degradation, comprising:

(i) providing at least one isolated microbial strain from excreta;

(ii) providing at least one lactic acid and/or acetic acid producing microbial strain;

(iii) providing at least one yeast strain;

(iv) providing at least one Bacillus sp., and

(v) combining the microbial strains from (i) to (iv) to produce a microbial consortium for waste degradation.

In a second aspect, the present invention provides a microbial consortium or a mixed microbial composition comprising:

(i) at least one isolated microbial strain from excreta;

(ii) at least one lactic acid and/or acetic acid producing microbial strain;

(iii) at least one yeast strain; and

(iv) at least one Bacillus sp.

In a third aspect, the present invention provides a product for waste degradation including the microbial consortium or the mixed microbial composition in accordance with the second aspect.

In a fourth aspect, the present invention provides a waste degradation method. The method includes providing one of the microbial consortium or the mixed microbial composition in accordance with the second aspect and the product for waste degradation in accordance with the third aspect; mixing the one of the microbial consortium or the mixed microbial composition and the product for waste degradation; and biodegrading the waste with the one of the microbial consortium or the mixed microbial composition and the product for waste degradation.

Other aspects and advantages of the invention will become apparent from the following detailed description, taken in conjunction with the accompanying drawings, illustrating by way of example the principles of the invention.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a photograph showing an immobilized microbial consortium that is ready for use.

FIG. 2 is a photograph showing human excreta added to an immobilized microbial consortium in a biotoilet cell at 0 hours (h).

FIG. 3 is a photograph showing complete degradation of the human excreta by the immobilized microbial consortium after 24 h.

### DETAILED DESCRIPTION

### Definitions

As used herein, “biodegrade” means to break down or decompose by biological processes. Thus, the process of decomposing an organic material by contacting the material with bacteria is an example of biodegradation.

As used herein, the term “composting” or “compostable” refers to the biodegradation or decomposition of organic matter and is carried out by various microorganisms, including bacteria, fungi and the like. The product of composting is compost which comprises a mixture of decaying organic matter. Compost may be used as a fertilizer.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

As used herein, “microorganism” is understood to refer to any microscopic organism including a eukaryote, a prokaryote or a virus; further including, but not limited to, a bacterium (either gram-positive, gram-negative or gram-variable), a fungus, a virus, a protozoan, algae and reproductive forms thereof including cysts and spores. With respect to bacteria, for example, it encompasses mycoplasmas, rickettsiae, and chlamydiae, which replicate within eukaryotic cells, as well as those bacteria which do not. The term “microorganism” may be used interchangeably with “microbial organism” and “microbe”.

As used herein, the term “isolated” as applied to a microorganism refers to a microorganism which has been removed and/or purified from an environment in which it naturally occurs. As such, an “isolated strain” of a microbe as used herein is a strain that has been removed and or purified from its natural milieu. Thus, an “isolated microorganism” does not include one residing in an environment in which it naturally occurs. Further, the term “isolated” does not necessarily reflect the extent to which the microbe has been purified. A “substantially pure culture” of the strain of microbe refers to a culture which contains substantially no other microbes than the desired strain or strains of microbe. In other words, a substantially pure culture of a strain of microbe is substantially free of other contaminants, which can include microbial contaminants as well as undesirable chemical contaminants. Further, as used herein, a “biologically pure” strain is intended to mean the strain separated from materials with which it is normally associated in nature. Note that a strain associated with other strains, or with compounds or materials that it is not normally found with in nature, is still defined as “biologically pure”. A monoculture of a particular strain is, of course, “biologically pure”. As used herein, the term “enriched culture” of an isolated microbial strain refers to a microbial culture that contains more than 50%, 60%, 70%, 80%, 90%, or of the isolated strain.

As used herein, a microbial consortium refers to a mixed population of two or more microbial strains. Typically, the microbial strains may form naturally or are combined together and achieve a specific purpose. For example, the microbial consortium of the present invention in combination is for biodegradation.

As used herein, the terms “organic matter” (used interchangeably with organic material) encompass any material comprising carbon including both fossilised and non-fossilised materials. Non-limiting examples of organic matter include biomass, lignocellulosic matter, and hydrocarbon-containing materials (e.g. lignite, oil shale and peat).

As used herein, “waste comprising organic matter” includes biological waste, manure, green waste, municipal waste, sewage, food and agricultural waste, and industrial organic waste. Manures can include manure produced by humans and various animals, including farm animals, such as, cows, sheep, horses, pigs, goats, rabbits, and poultry such as chickens, turkeys, and ducks. Green waste can include a variety of substrates from several sources such as yard wastes including grass clippings, tree, brush and hedge trimmings, and leaves, as well as domestic and commercial food waste. Municipal waste can include residential and commercial refuse, such as paper, wood, food and yard wastes. Waste that may be biodegraded or composted can be separated from commingled non-biodegradable matter. Sewage sludge can be used as a source of organic waste.

### DESCRIPTION

According to a first aspect, the present invention provides a method of producing a product for waste degradation, comprising:

(i) providing at least one isolated microbial strain from excreta;

(ii) providing at least one lactic acid and/or acetic acid producing microbial strain;

(iii) providing at least one yeast strain;

(iv) providing at least one Bacillus sp., and

(v) combining the microbial strains from (i) to (iv) to produce a microbial consortium for waste degradation.

In a second aspect, the present invention provides a microbial consortium or a mixed microbial composition comprising:

(i) at least one isolated microbial strain from excreta;

(ii) at least one lactic acid and/or acetic acid producing microbial strain;

(iii) at least one yeast strain; and

(iv) a least one Bacillus sp.

In a third aspect, the present invention provides an isolated microbial strain from excreta.

In a fourth aspect, the present invention provides a substantially pure culture of an isolated microbial strain from excreta.

Any isolated microbial strain from excreta may be contemplated for preparing a microbial consortium, for a microbial consortium or an isolated microbial strain from excreta of the present invention. For example, the isolated microbial strain(s) from excreta may be selected from a group consisting of Paenibacillus sp., Bacillus sp. and Pediococcus sp.

The Paenibacillus sp. may comprise Paenibacillus sp. DSMZ 32878. The Bacillus sp. may comprise Bacillus sp. DSMZ 32879. The Pediococcus sp. may comprise Pediococcus acidilactici. In particular, the Pediococcus sp. may comprise Pediococcus acidilactici DSMZ 32880. The DSMZ numbers throughout this specification are the deposit numbers with Leibniz-Institute DSMZ-German Collection of Microorganisms (a Budapest treaty international deposit authority).

It will be appreciated that the isolated microbial strains from excreta for any aspect of the invention may consist of Paenibacillus sp. DSMZ 32878, Bacillus sp. DSMZ 32879 and/or Pediococcus acidilactici DSMZ 32880.

Accordingly, the invention provides an isolated strain of Paenibacillus sp. DSMZ 32878. The invention also provides an isolated strain of Bacillus sp. DSMZ 32879. The invention further provides an isolated strain of Pediococcus acidilactici DSMZ 32880.

Any lactic acid and/or acetic acid producing microbial strain may be contemplated for preparing a microbial consortium or for a microbial consortium of the present invention. The lactic acid and/or acetic acid producing microbial strain may be an isolated lactic acid and/or acetic acid producing bacteria. For example, the lactic acid and/or acetic acid producing microbial strain(s) may be selected from a group consisting of Acetobacter sp., Lactobacillus sp. and Lactococcus sp.

The Acetobacter sp. may comprise Acetobacter pasteurianus. The Lactococcus sp. may comprise Lactococcus lactis. It will be appreciated that any commercially available Acetobacter sp, Acetobacter pasteurianus, Lactobacillus sp., Lactococcus sp, and Lactococcus lactis may be used.

It will be appreciated that in an embodiment, the lactic acid and/or acetic acid producing microbial strains may consist of Acetobacter pasteurianus, Lactobacillus sp. and/or Lactococcus lactis.

Advantageously, the at least one lactic acid and/or acetic acid producing microbial strain that is/are added creates an acidic environment to inhibit harmful microbes and odour, as well as facilitates biodegradation.

Any yeast strain may be contemplated for preparing a microbial consortium or for a microbial consortium of the present invention. The yeast strain may be an isolated yeast strain. For example, the yeast strain may be Saccharomyces sp. The Saccharomyces sp. may comprise Saccharomyces cerevisiae. It will be appreciated that any Saccharomyces cerevisiae may be used, for example, from commercially available Baker's yeast.

Advantageously, the at least one yeast strain that is added reduces unpleasant odour.

Any Bacillus sp. may be contemplated for preparing a microbial consortium or for a microbial consortium of the present invention. The Bacillus sp. may be isolated. The at least one Bacillus sp. of (iv) for the first and second aspects of the invention may comprise Bacillus subtilis. For example, the at least one Bacillus sp. of (iv) may comprise Bacillus subtilis DSMZ 32881. In an alternative embodiment, any commercially available Bacillus sp. may be used.

Advantageously, the at least one Bacillus sp. that is added produces certain essential enzymes for biodegradation of human excreta and, more particularly, enhances production of important enzymes for fast biodegradation of excreta.

Further advantageously, all strains (i) to (iv) of the first and second aspects of the invention are safe for use.

Each of the isolated microbial strain(s) from excreta, the at least one lactic acid and/or acetic acid producing microbial strain(s), the at last one yeast strain and the at least one Bacillus sp. may be cultured separately before combining to produce the microbial consortium for waste degradation. In an alternative embodiment, the isolated microbial strain(s) from excreta, the at least one lactic acid and/or acetic acid producing microbial strain(s), the at last one yeast strain and the at least one isolated Bacillus sp. may be combined and co-cultured to produce the microbial consortium for waste degradation.

The method of producing a product for waste degradation may further comprise immobilizing the microbial consortium on a solid medium. Any suitable solid medium may be used for immobilizing the substantially pure culture, the microbial consortium or the mixed microbial composition. The solid medium is preferably cheap and biodegradable. Examples of a suitable solid medium include, but are not limited to, sawdust, spent grains or a solid medium derived from oil palm industry such as, for example, empty fruit bunch of oil palm.

The present invention includes a product for waste degradation comprising the microbial consortium or the mixed microbial composition as described herein.

The present invention also includes a waste degradation method, comprising providing one of the microbial consortium or the mixed microbial composition and the product for waste degradation as described herein, mixing the one of the microbial consortium or the mixed microbial composition and the product for waste degradation with waste; and biodegrading the waste with the one of the microbial consortium or the mixed microbial composition and the product for waste degradation. The waste may be biodegraded at a temperature of between about 20 degrees Celsius (° C.) and about 50° C., more preferably, between about 30° C. and about 50° C. The waste may include excreta.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Green and Sambrook, Molecular Cloning: A Laboratory Manual, Cold Springs Harbor Laboratory, New York (2012).

### Example 1

### Screening of Strains from Human Excreta (Faeces)

Screening was done via two methods.

In the first method, a small quantity of human excreta (faeces) (10 mg) was placed in culture tubes of 5 mL of a tryptic soy broth (TSB, Sigma Aldrich, USA) and grown overnight at 30° C. and 200 rpm. The resultant culture was streaked on tryptic soy agar (TSA, Sigma Aldrich, USA) and grown overnight at 30° C. Colonies on TSA were sub-cultured subsequently for 16S ribosomal DNA (rDNA) analysis.

In the second method, 100 mg of human excreta (faeces) was placed in culture flasks of 30 mL of an Yeast Extract-Peptone-Glycerol (YPG) medium containing 4% ethanol (10 g/L yeast extract, 20 mL/L glycerol, 20 g/L peptone) and grown overnight at 30° C. and 200 rpm. Serial dilution of the culture was performed before spreading it on Yeast Extract-Glucose-Salt (YGS) agar (20 g/L glucose, 20 g/L yeast extract, 5 g/L NaCl, 2 g/L KH₂PO₄, 15 g agar). The agar plates were grown overnight at 30° C. Single colonies on YGS agar were subsequently sub-cultured and used for 16S ribosomal DNA (rDNA) analysis.

All isolates from both methods were grown in their respective media and about 2 mL of cell culture was collected for each strain, centrifuged and drained to obtain cell pellet for identification. Genomic DNA of each isolate was extracted using DNeasy Blood and Tissue Kit (Qiagen, Netherlands). The 16S rDNA was amplified using Phusion High Fidelity DNA Polymerase (Thermo Fisher Scientific, USA) and universal primers (Fwd 5′-AGAGTTTGATCATGGCTCAG-3′ (SEQ ID NO: 1) and Rev 5′-AAGGAGGTGATCCAGCCGCA-3′ (SEQ ID NO: 2)). PCR was performed on G-Storm 2 Thermal Cycler (G Storm, UK) under the following conditions: an initial denaturation at 98° C. for 30 s followed by 30 cycles of denaturation at 98° C. for 30 s, annealing at 55° C. for 20 s and extension at 72° C. for 45 s. The final extension was conducted at 72° C. for 10 min. The resultant PCR products were electrophoresized on 1% (w/v) agarose gel at 120 V, 30 min, gel purified using Qiagen QIAquick Gel Extraction Kit (Qiagen, Netherlands) and sequenced. A homology search of the isolated 16S rDNA was done using nBLAST on NCBI website and analyzed for species identification. Finally, three biosafety Level 1 isolates were selected for making the microbial consortium.

TABLE 1
Selected isolates from solid human excreta (faeces)

| Isolate | Identity |
|---|---|
| BT-2 | Paenibacillus sp. |
| | (DSMZ 32878) |
| BT-3 | Bacillus sp. |
| | (DSMZ 32879) |
| BT-28 | Pediococcus |
| | acidilactici |
| | (DSMZ 32880) |

### Example 2

### Screening of Acetic Acid and Lactic Acid Producing Microbes from Commercial Sources

Commercial acetic acid and lactic acid microbial powder were purchased from Taobao (Alibaba Group, China). For isolation of acetic acid strains, a small quantity of the powder (10 mg) was placed in culture tubes of 5 mL YPG medium supplemented with ethanol (10 g/L yeast extract, 20 g/L peptone, 20 mL/L glycerol and 50 mL/L ethanol) and grown for 2 to 3 days at 30° C. and 200 rpm. The resultant culture was streaked on solid agar of the same medium and grown for 2 days at 30° C. Colonies were subsequently sub-cultured for 16S ribosomal DNA (rDNA) analysis. For isolation of lactic acid strains, a small quantity of the powder (10 mg) was placed in culture tubes of 5 mL MRS broth (Sigma Aldrich, USA) and grown overnight at 30° C. and 200 rpm. The resultant culture was streaked on MRS agar plate and grown for 1 day at 30° C. All the isolated colonies for acetic and lactic acid microbes were identified using procedures similar to Example 1 above with the exception of yeast strains which required a different set of primers (F-566 5′-CAGCAGCCGCGGTAATTCC-3′ (SEQ ID NO: 3) and R-1200 5′-CCCGTGTTGAGTCAAATTAAGC-3′ (SEQ ID NO: 4)). The identities of the strains are shown in Table 2.

TABLE 2
Identification of acetic acid- and lactic
acid-producing microbes and yeast strain

| Isolate | Identity |
|---|---|
| AA2 | Saccharomyces cerevisiae |
| AA8 | Acetobacter pasteurianus |
| Lacid 3-1 | Lactobacillus sp. |
| Lacid 12 | Lactococcus lactis |

### Example 3

### Development of WM8 for Human Excreta Composting

A new microbial consortium WM8 was developed by mixing cultures of the three (3) isolates from human excreta (Table 1), the four (4) isolates from commercial sources (Table 2) and one Bacillus subtilis (DSMZ 32881) isolated from food waste. All the strains were grown in their respective media and temperatures as listed in Table 3 below. Starter cultures for all strains were prepared in 20-50 mL of media in shake flasks, with colonies on agar plates directly inoculated into respective media and grown overnight at 200 rpm and preferred temperatures. Thereafter, the starter cultures were used to inoculate into 100 mL of the respective media at 5-10% (v/v) and grown for 2 days at 200 rpm and preferred temperatures. To concentrate the cultures for higher cell density, cultures were transferred into 500 mL centrifuge bottles and centrifuged for 5 min at 10° C. and 8,000 rpm. Half the supernatant was discarded and the remaining content of each culture was mixed thoroughly.

TABLE 3
Media and temperatures used for cultivating
individual isolated strains

| Isolated Strain | Medium | Temperature |
|---|---|---|
| BT-2 | QXM (50 g/L dextrose, 20 g/L yeast | 30° C. |
| (DSMZ 32878) | extract, 2 g/L (50 g/L dextrose, | |
| | 20 g/L yeast extract, 2 g/L | |
| BT-3 | TSB (Tryptic soy broth, Sigma | 30° C. |
| (DSMZ 32879) | Aldrich) | |
| BT-28 | MRS (MRS broth, Sigma Aldrich) | 37° C. |
| (DSMZ 32880) | | |
| AA2 | YPD (10 g/L yeast extract, 20 g/L | 30° C. |
| | peptone andv20 g/L dextrose) | |
| AA8 | YGC (10 g/L Yeast extract, 50 g/L | 28° C. |
| | D-Glucose, 20 g/L CaCO3) | |
| Lacid 3-1 | MRS (MRS broth, Sigma Aldrich) | 30° C. |
| Lacid 12 | BHI (Brain heart infusion broth, | 37° C. |
| | Oxoid) | |
| WM3-25C | LB (Luria Bertani broth, Sigma | 30° C. |
| Bacillus subtilis | Aldrich) | |
| (DSMZ 32881) | | |

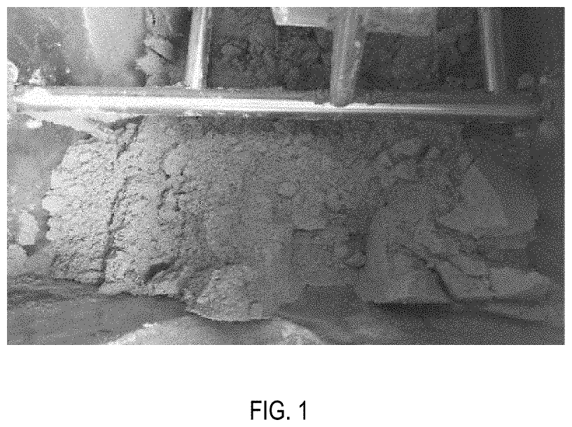

The mixed cell culture was immobilized by adding sawdust until no free water was visible. The immobilized cells (about 5 kg) were stabilized at 30° C. for 24 h with continuous rotation before becoming ready for use as shown in FIG. 1.

### Example 4

### Composting of Human Excreta by the Immobilized Microbial Consortium

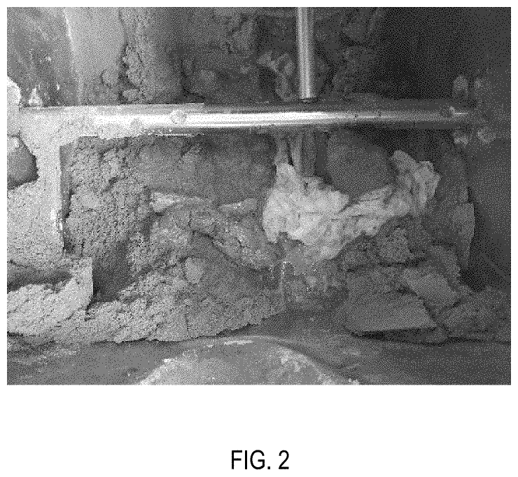

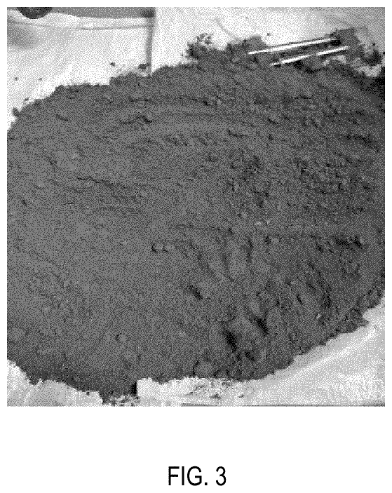

Human excreta (faeces 180 g and urine 50 g) was added into the immobilized microbial consortium in a biotoilet cell as shown in FIG. 2 and the mixture was well mixed by rotation and maintained at 20-50° C. After 1 h, the human excreta was almost invisible. After 24 h, 100% of the human waste was degraded and the compost looked like dry powder fertilizer as shown in FIG. 3.

### Discussion

A new microbial consortium WM8 was developed by isolating microbes from human excreta and combining the isolated microbial strains with selected additional microbial strains. The microbial consortium may be immobilized on cheap solid support to enhance the stability of the microbial consortium for commercial usage. The human excreta (faeces+urine) was almost completely degraded after treatment using the immobilized microbial consortium within 24 h at 20-50° C.

The microbial consortium may be composed of seven (7) bacteria strains and one (1) yeast strain. These may be obtained from one or more of toilet samples, food resources or commercial sources and may all be Biosafety Level 1 strains.

The microbial consortium makes it feasible to compost human excreta without having to separate urine from faeces. The microbial consortium is able to compost human excreta (faeces and urine) more efficiently than commercially available microbial consortia with shortened composting times of less than 24 h, lower composting temperatures of from 20-50° C. and achieving higher reduction rates of more than 90% of the human excreta. Advantageously, this reduces composting time and energy consumption and also improves composting efficiency, thereby benefiting the environment and the economy. Further advantageously, the microbial consortium may be used in the absence of flushing water for biotoilet applications.

While preferred embodiments of the invention have been illustrated and described, it will be clear that the invention is not limited to the described embodiments only. Numerous modifications, changes, variations, substitutions and equivalents will be apparent to those skilled in the art without departing from the scope of the invention as described in the claims.

1. A method of producing a product for waste degradation, the method comprising:
(i) providing at least one isolated microbial strain from excreta;
(ii) providing at least one lactic acid and/or acetic acid producing microbial strain;
(iii) providing at least one yeast microbial strain;
(iv) providing at least one Bacillus sp. microbial strain, and
(v) combining the microbial strains from (i) to (iv) to produce a microbial consortium for waste degradation.

2. The method according to claim 1, wherein the strain(s) from (i) is/are selected from a group consisting of Paenibacillus sp., Bacillus sp. and Pediococcus sp.

3. The method according to claim 2, wherein the Paenibacillus sp. comprises Paenibacillus sp. DSMZ 32878.

4. The method according to claim 2, wherein the Bacillus sp. comprises Bacillus sp. DSMZ 32879.

5. The method according to claim 2, wherein the Pediococcus sp. comprises Pediococcus acidilactici.

6. The method according to claim 5, wherein the Pediococcus sp. comprises Pediococcus acidilactici DSMZ 32880.

7. The method according to claim 2, wherein the strains from (i) consist of Paenibacillus sp. DSMZ 32878, Bacillus sp. DSMZ 32879 and Pediococcus acidilactici DSMZ 32880.

8. The method according to claim 1, wherein the strain(s) from (ii) is/are selected from a group consisting of Acetobacter sp., Lactobacillus sp. and Lactococcus sp.

9. The method according to claim 8, wherein the Acetobacter sp. comprises Acetobacter pasteurianus.

10. The method according to claim 8, wherein the Lactococcus sp. comprises Lactococcus lactis.

11. The method according to claim 8, wherein the strains from (ii) consist of Acetobacter pasteurianus, Lactobacillus sp. and Lactococcus lactis.

12. The method according to claim 1, wherein the at least one yeast strain from (iii) comprises Saccharomyces sp.

13. The method according to claim 12, wherein the Saccharomyces sp. comprises Saccharomyces cerevisiae.

14. The method according to claim 1, wherein the at least one Bacillus sp. from (iii) comprises Bacillus subtilis.

15. The method according to claim 14, wherein the at least one Bacillus sp. from (iii) comprises Bacillus subtilis DSMZ 32881.

16. The method according to claim 1, wherein each of microbial strains in (i), (ii), (iii) and (iv) is cultured separately before combining to produce the microbial consortium for waste degradation.

17. The method according to claim 1, wherein the microbial strains in (i), (ii), (iii) and (iv) are combined and co-cultured to produce the microbial consortium for waste degradation.

18. The method according to claim 1, further comprising immobilizing the microbial consortium on a solid medium.

19. The method according to claim 18, wherein the solid medium is sawdust, spent grains or derived from empty fruit bunch of oil palm.

20. A microbial consortium or a mixed microbial composition comprising:
(i) at least one isolated microbial strain from excreta;
(ii) at least one lactic acid and/or acetic acid producing microbial strain;
(iii) at least one yeast strain; and
(iv) at least one Bacillus sp.

21. The microbial consortium or mixed microbial composition according to claim 20, wherein the strain(s) from (i) is/are selected from a group consisting of Paenibacillus sp., Bacillus sp. and Pediococcus sp.

22. The microbial consortium or mixed microbial composition according to claim 21, wherein the Paenibacillus sp. comprises Paenibacillus sp. DSMZ 32878.

23. The microbial consortium or mixed microbial composition according to claim 21, wherein the Bacillus sp. comprises Bacillus sp. DSMZ 32879.

24. The microbial consortium or mixed microbial composition according to claim 21, wherein the Pediococcus sp. comprises Pediococcus acidilactici.

25. The microbial consortium or mixed microbial composition according to claim 24, wherein the Pediococcus sp. comprises Pediococcus acidilactici DSMZ 32880.

26. The microbial consortium or mixed microbial composition according to claim 21, wherein the strains from (i) consist of Paenibacillus sp. DSMZ 32878, Bacillus sp. DSMZ 32879 and Pediococcus acidilactici DSMZ 32880.

27. The microbial consortium or mixed microbial composition according to claim 20, wherein the strain(s) from (ii) is/are selected from a group consisting of Acetobacter sp., Lactobacillus sp. and Lactococcus sp.

28. The microbial consortium or mixed microbial composition according to claim 27, wherein the Acetobacter sp. comprises Acetobacter pasteurianus.

29. The microbial consortium or mixed microbial composition according to claim 27, wherein the Lactococcus sp. comprises Lactococcus lactis.

30. The microbial consortium or mixed microbial composition according to claim 27, wherein the strains from (ii) consist of Acetobacter pasteurianus, Lactobacillus sp. and Lactococcus lactis.

31. The microbial consortium or mixed microbial composition according to claim 20, wherein the at least one yeast strain from (iii) comprises Saccharomyces sp.

32. The microbial consortium or mixed microbial composition according to claim 31, wherein the Saccharomyces sp. comprises Saccharomyces cerevisiae.

33. The microbial consortium or mixed microbial composition according to claim 20, wherein the at least one Bacillus sp. from (iv) comprises Bacillus subtilis.

34. The microbial consortium or mixed microbial composition according to claim 33, wherein the at least one Bacillus sp. from (iv) comprises Bacillus subtilis DSMZ 32881.

35. The microbial consortium or the mixed microbial composition according to claim 20 immobilized with a solid medium.

36. The microbial consortium or the mixed microbial composition according to claim 35, wherein the solid medium is sawdust, spent grains or derived from empty fruit bunch of oil palm.

37. A product for waste degradation comprising the microbial consortium or the mixed microbial composition according to claim 20.

38. A waste degradation method, comprising:
providing one of a microbial consortium, or a mixed microbial composition, or a product for waste degradation comprising the microbial consortium or the mixed microbial composition;
mixing with waste the one of the microbial consortium or the mixed microbial composition or the product for waste degradation; and
biodegrading the waste with the one of the microbial consortium or the mixed microbial composition or the product for waste degradation,
wherein the microbial consortium and the mixed microbial composition comprise: (i) at least one isolated microbial strain from excreta; (ii) at least one lactic acid and/or acetic acid producing microbial strain; (iii) at least one yeast strain; and (iv) at least one Bacillus sp.

39. The waste degradation method according to claim 38, wherein the waste is biodegraded at a temperature of from about 20 degrees Celsius (° C.) to about 50° C.

40. The waste degradation method according to claim 38, wherein the waste comprises excreta.


SEQUENCE LISTING (4 sequences)

SEQ ID NO: 1 - DNA, 20 bp, Artificial Sequence
  synthetic primer
agagtttgatcatggctcag

SEQ ID NO: 2 - DNA, 20 bp, Artificial Sequence
  synthetic primer
aaggaggtgatccagccgca

SEQ ID NO: 3 - DNA, 19 bp, Artificial Sequence
  synthetic primer
cagcagccgcggtaattcc

SEQ ID NO: 4 - DNA, 22 bp, Artificial Sequence
  synthetic primer
cccgtgttgagtcaaattaagc